(19) United States
(12) Patent Application Publication
(10) Pub. No.: US 2026/0080714 A1
(43) Pub. Date: Mar. 19, 2026

(54) TRANSFORMER-BASED AUDIO-VISUAL AUTISM RECOGNITION SYSTEM BASED ON FAMILY OBSERVATION SCHEDULE

(71) Applicant: The George Washington University, Washington, DC (US)

(72) Inventors: Chung Hyuk Park, Falls Church, VA (US); Zhenhao Zhao, Washington, DC (US)

(73) Assignee: The George Washington University, Washington, DC (US)

(21) Appl. No.: 19/266,869

(22) Filed: Jul. 11, 2025

Related U.S. Application Data
(60) Provisional application No. 63/669,976, filed on Jul. 11, 2024.

(51) Int. Cl. G06V 40/20; A61B 5/00; G06V 10/26; G06V 20/40; G10L 15/04; G10L 25/18; G10L 25/57; G10L 25/63

(52) U.S. Cl. CPC G06V 40/20; A61B 5/4803; G06V 10/26; G06V 20/46; G06V 20/49; G10L 15/04; G10L 25/18; G10L 25/57; G10L 25/63

(57) ABSTRACT

A behavior recognition system for analyzing an audio-video signal to detect challenging behaviors in autism via behavioral features. The system includes a processor configured to segment the audio-video signal into clips of audio data and video data, each of said clips having a predefined duration and annotated with interaction styles. The processor samples and preprocesses the audio data and video data of said clips to provide square video patches and square audio patches. The processor tokenizes the square video patches to embed video positional information and video modality information, and tokenize the square audio patches to embed audio positional information and audio modality information. And, the processor predicts behaviors based on the tokenized square video patches and the tokenized square audio patches.

### RELATED APPLICATION

This application claims the benefit of priority of U.S. Provisional Application No. 63/669,976, filed on Jul. 11, 2024, including the references cited therein, the entire content of which is relied upon and incorporated herein by reference in its entirety.

### GOVERNMENT LICENSE RIGHTS

This invention was made with Government support under National Science Foundation (NSF) under Grant #1846658. The Government has certain rights in this invention.

### BACKGROUND

Challenging behaviors in children with autism is a serious clinical condition, oftentimes leading to aggression or self-injurious actions. The Family Observation Schedule 2nd Edition (FOS-II) is an intensive and fine-grained scale used to observe and analyze the behaviors of individuals with autism, which facilitates the diagnosis and monitoring of autism severity. Previous AI-based approaches for automated behavior analysis in autism often focused on predicting facial expressions and body movements without generating a clinically meaningful scale, mostly utilizing visual information.

Autism Spectrum Disorder (ASD), or autism, is a life-long neuro-developmental condition. The increasing prevalence of ASD among children in the United States has become a significant developmental issue. Over the past decades, the rate has been steadily rising, with 1 in 36 children now diagnosed with autism. Individuals with ASD, or autistic individuals, experience difficulties in communication and social interaction, exhibit restricted interests, and engage in repetitive behaviors. These characteristics impact their daily activities and social functioning across various settings such as school, work, and other areas of life.

One of the more clinically important characteristics with autistic individuals is the challenging behaviors (CBs), such as self-injurious behaviors, aggression and disruptive behaviors. These CBs not only hinder social interaction but also frequently result in critical health implications for the individuals themselves or others. Despite their clinical importance, tracking these behaviors in daily settings remains a significant challenge. Currently, monitoring CBs primarily relies on regular clinical evaluations conducted in office settings, which imposes considerable burdens and restrictions on families of autistic individuals. Moreover, this approach is cost-prohibitive and unsuitable for long-term continuous observation. The sporadic nature of certain episodes may further lead to discrepancies between diagnostic outcomes and actual behavioral patterns. Therefore, developing automated tools capable of analyzing the interactive behaviors between autistic children and their caregivers is not only beneficial for the diagnosis and treatment of children but also essential for reducing the burden on caregivers. Additionally, such tools would facilitate long-term monitoring, enabling more accurate diagnoses and a better understanding of behavioral trends over time.

One of the clinical measures that has been established for rigorous and fine-grained coding of children behaviors is the Family Observation Schedule-Second Version (FOS-II) [9], which is a direct observation tool designed to assess parent-child interactions across various contexts. In autism research, FOS-II is frequently utilized in both clinical and research settings to identify and evaluate parent-child interactions, particularly in relation to CBs. This tool provides valuable insights for developing interventions and support strategies for autistic children by examining their social contexts and dynamics [1]. Currently, FOS-II data is manually encoded by trained observers through video interactions between autistic children and their caregivers, a process that is both time-consuming and labor-intensive.

U.S. Pat. No. 10,687,751 disclose a system to enhance diagnosis of disorder through artificial intelligence and mobile health technologies without compromising accuracy. That patent targets a general diagnosis framework on all aspects of NDD but with no clinical validity. US Patent Publ. No. 2024407685 discloses a method and apparatus for supporting autism spectrum disorder diagnosis based on artificial intelligence, but does not specifically deal with CBs but mainly provides a general tool for behavior monitoring.

### SUMMARY

A transformer-based audio-visual autism recognition system is provided based on the family observation schedule system. The system includes an automated FOS-II encoding algorithm suitable for clinical settings to significantly reduce the workload for clinicians and researchers, ultimately benefiting many autistic children and their families. The automated tools apply a multimodal sensor-based approach with artificial intelligence (AI).

These and other objects of the disclosure, as well as many of the intended advantages thereof, will become more readily apparent when reference is made to the following description, taken in conjunction with the accompanying drawings. This summary is not intended to identify all essential features of the claimed subject matter, nor is it intended for use in determining the scope of the claimed subject matter. It is to be understood that both the foregoing general description and the following detailed description are exemplary and are intended to provide an overview or framework to understand the nature and character of the disclosure.

### BRIEF DESCRIPTION OF THE FIGURES

The accompanying drawings are incorporated in and constitute a part of this specification. It is to be understood that the drawings illustrate only some examples of the disclosure and other examples or combinations of various examples that are not specifically illustrated in the figures may still fall within the scope of this disclosure. Examples will now be described with additional detail through the use of the drawings, in which:

FIG. 1A shows the system in accordance with a non-limiting example embodiment of the present disclosure.

FIG. 1B is a subset of IS examples, accompanied by a single frame from the corresponding videos, is displayed. All images have been anonymized to safeguard the privacy and confidentiality of the participants.

FIG. 2. The coding sheet of the annotation.

FIG. 3. Comparison of three spatial-temporal attention approaches.

FIG. 4. The data preprocessing and tokenization.

FIG. 5. The pretrained structure of the CAV-MAE. We followed the original CAV-MAE paper [2], used reconstruction loss and contrastive loss for the pretrained.

FIG. 6. The FOS-II decision neural network: AV-FOS.

FIG. 7. The performance and time cost comparison.

FIG. 8. The confusion matrix for different algorithms.

FIG. 9. The attention map for the joint perception layer

### DETAILED DESCRIPTION

In describing the present disclosure illustrated in the drawings, specific terminology is resorted to for the sake of clarity. However, the present disclosure is not intended to be limited to the specific terms so selected, and it is to be understood that each specific term includes all technical equivalents that operate in a similar manner to accomplish a similar purpose.

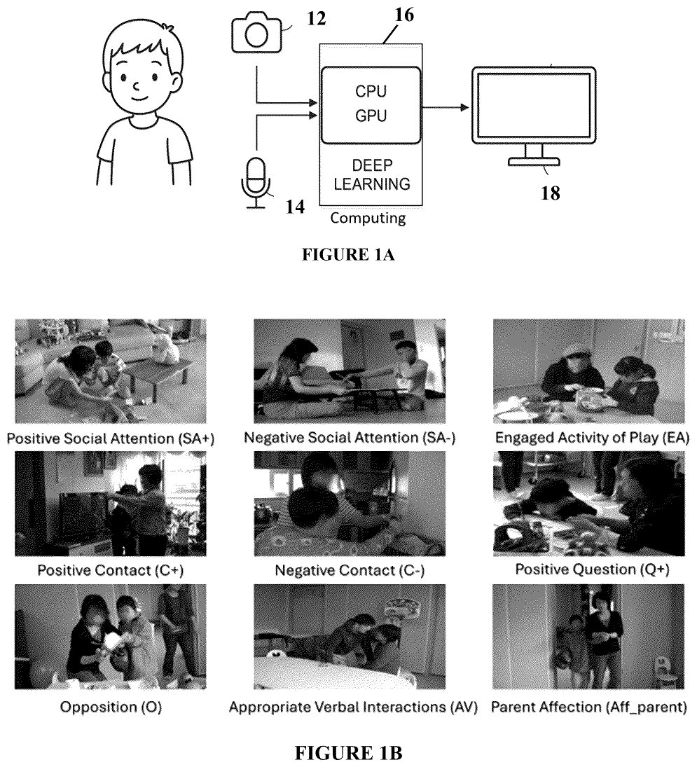

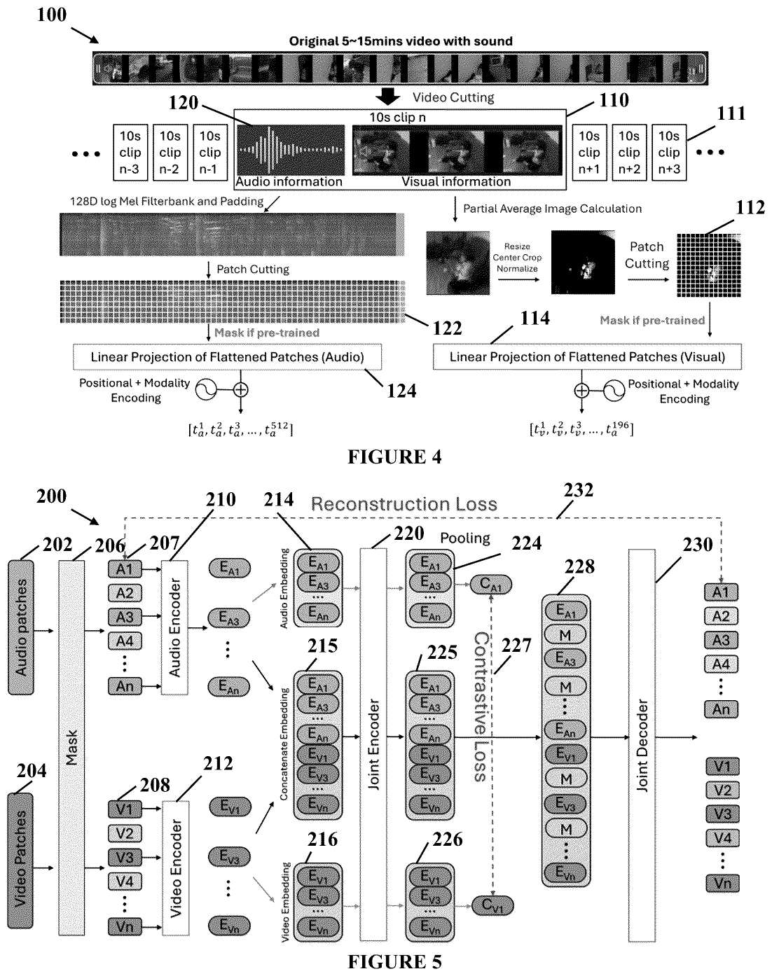

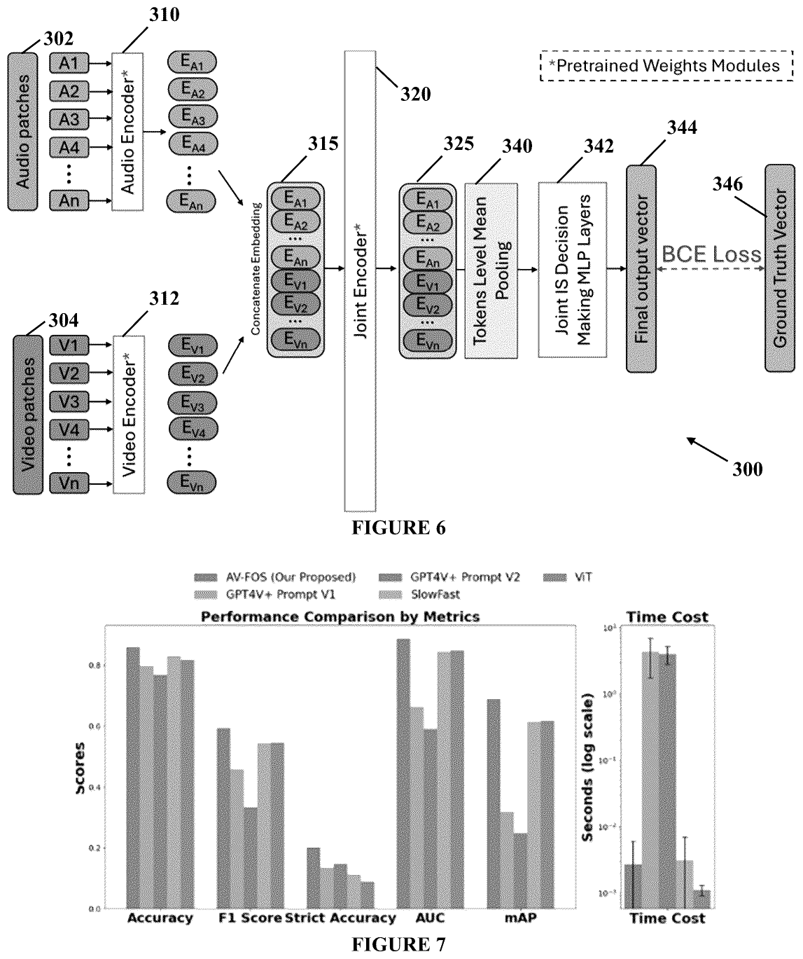

Turning to the drawings, FIG. 1A presents an overarching description of a non-limiting example embodiment of the system of the disclosure. At its center is the deep learning algorithm, the present AV-FOS, which processes video-modality information from an image detector such as the camera 12 and audio-modality information from an audio detector such as a microphone 14, using a processing device 16 (e.g., both a CPU and a GPU). The data-preprocessing phase of this model is illustrated in FIG. 4, and the inference phase of the deep learning model is shown in FIG. 6. FIG. 5, by contrast, depicts the architecture employed during the model's self-supervised pretraining and is not used in post-deployment applications. Thus, FIG. 6 is the deep learning structure of AV-FOS model. Before the model feeds into the AV-FOS model, the data is preprocessed using the structure shown in FIG. 4. Output and reports are displayed on a monitor 18. Data (including, for example, algorithms, AV-FOS model, FOS-II dataset, image data, audio data, training data, and processed data) can be stored in a memory (e.g., database) that is in communication with the processing device 16.

FIG. 6 shows a transformer-based audio-visual autism recognition system 10. The system 10 utilizes an automated FOS-II encoding algorithm to conduct a family observation schedule (FOS-II) to assess autism in individuals. It is suitable for clinical settings to significantly reduce the workload for clinicians and researchers, ultimately benefiting many autistic children and their families. The automated tools apply a multimodal sensor-based approach with artificial intelligence (AI), such as camera and microphones (12 and 14 in FIG. 1) to get audio and video data as inputs.

In recent years, Transformer-based multimodal models have demonstrated strong capabilities across various video understanding tasks [3], [4]. However, these transformer-based models heavily rely on extensive enterprise-level computational resources and large datasets for training, while the clinical observational data of autistic children is typically not easily accessible due to privacy issues and the size of the data is small.

To address these challenges, the system first includes a high-quality FOS-II dataset, meticulously annotated by experts. This dataset comprises nearly 25 hours of videos featuring autistic children, with Interaction Styles (IS) from FOS-II annotated every 10 seconds. This dataset is highly suitable for both supervised and unsupervised learning in deep learning models, facilitating future research on deep learning algorithms for autistic children. Secondly, the system comprises an audio-visual transformer-based model (AV-FOS) for recognizing interaction styles in autistic children, which features relatively manageable computational requirements and real-time inference speed. The AV-FOS model was trained and tested on our FOS-II dataset. As a baseline, we compared it with the enterprise level model (GPT-4V [5]) combined with prompt engineering. As comparison models, we applied our dataset to two vision-based behavior understanding AI models SlowFast Networks [6] and vision transformer [7] and conducted an ablation study. Our AV-FOS model exhibited superior performance and inference speed compared to the baseline as well as comparison models.

### A. FOS-II Clinical Application and Case Study

The FOS-II is a highly validated coding system designed to capture negative behaviors and interaction styles of both children with ASD and their parents at 10-second intervals. It is widely recognized for its utility in observing challenging behaviors. For instance, in the study by Sander et al. [8], the FOS-II was employed to assess changes in children's problem behaviors and parent-child interactions before and after a behavioral parenting program. Mother and child behaviors were evaluated through a 30-minute video-recorded home observation, with coding performed at 10-second intervals using the FOS-II system. The study found a significant reduction in negative child behaviors in the intervention group compared to the control group, underscoring the value of FOS-II in quantifying behavioral changes.

Similarly, Pasalich et al. [9] utilized the FOS-II system to investigate the associations between callous-unemotional (CU) traits, conduct problems in children with ASD, and parental warmth/responsiveness. This study involved a 24-30 minute behavioral observation session that included free play and parental instruction activities. Parental warmth and responsiveness were coded using the FOS-II, highlighting how both ASD symptoms and CU traits significantly influenced child conduct problem severity and the quality of family relationships. These findings further demonstrate the versatility of the FOS-II system in capturing nuanced parent-child interaction dynamics.

However, in previous studies, FOS-II coding has predominantly relied on manual processes, which can be labor-intensive and time-consuming. Existing ASD behavior assessment services face limitations due to a shortage of specialists and restricted access to professional institutions, often imposing substantial financial and time burdens on families. The development of a deep learning model capable of automatically analyzing home-recorded videos and providing realtime assessment results could effectively address these challenges. Such a model would facilitate early detection of behavioral pattern changes or increases in specific challenging behaviors, enabling timely intervention and the development of tailored strategies to support affected families. The environment is uncontrolled daily environments from multiple homes, with user age and gender variations. And classification data includes over 14 dimensions, which makes the dataset highly non-linear and not suitable for traditional machine learning approaches, through the deep neural network's strong capacity to process non-linear data and learn intrinsic patterns and features from the complex data.

One technical difficulty was to process the multimodal input (the audio and vision/image data) at same time. To do so, the present system uses the multi-modal transformer structure of the FOS dataset which is a multi-modal classification dataset. Another technical difficulty has been with the lacking data, since the labeled data is insufficient. Accordingly, the present system utilizes self-supervised learning technique for the pretraining. Another technical difficulty has been to solve the visual time information perception problem. To do so, the present system utilizes averaged key frame attention.

### B. Multimodal Learning for Behavior Recognition

Multimodal behavior recognition is a highly active research field. Previous studies have focused on various aspects, such as emotion/behavior recognition using video and text information, or action recognition using various visual modalities like optical flow and skeleton tracking. However, the previous studies have limitations on the modality of inputs, limiting the bandwidth of contextual understanding. Thus, the present system focuses on recognizing behaviors of autistic children and their caregivers using audio and video modalities capable of providing fine-grained clinical explainability.

Noteworthy are the two AI-based multimodal models capable of audio+video understanding: Audio-Visual Masked Autoencoder (AV-MAE) [10] and Contrastive Audio-Visual Masked Autoencoder (CAV-MAE) [2], FIG. 5.

We adapt these state-of-the-art transformer-based approaches and provide a customized architecture (FIG. 6) advancing from AV-MAE and CAV-MAE to self-learn the clinical measures in FOS-II scale and provide explainable AI module on audio-visual inputs. Our AV-FOS model adapts similar pre-training algorithms as the CAV-MAE but adds new strategies to achieve supervised learning with fine-grained self-built clinical dataset. Furthermore, given the limited capacity of the CAV-MAE model to perceive visual temporal information, we address this limitation through targeted optimizations.

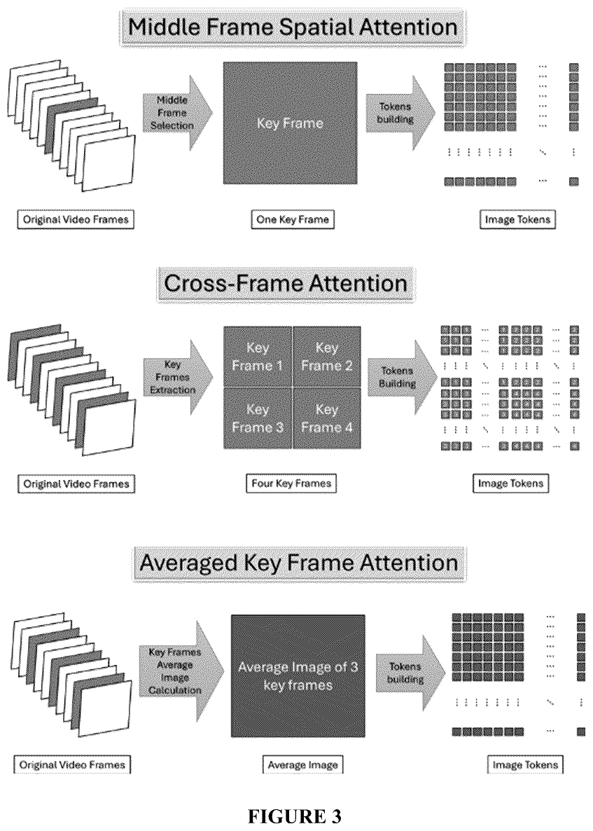

The optimization strategy is referred to here as “Averaged Key Frame Attention,” which is shown in FIG. 3. The system extracts one keyframe each from the first, middle, and final thirds of the video, compute their pixel-level average image, resize it to 224×224 pixels, and divide it into 196 square patches. The introduction of this paradigm enhances the model's temporal visual perception capabilities. FIG. 4 shows the data preprocessing where the Averaged Key Frame Attenuation is used in the model, at the “Partial average image calculation” step.

### C. Deep Learning-Based Autism Research

There has been extensive research utilizing deep learning techniques in studies of autistic children. Some studies focus on emotion recognition in autistic patients based on their facial expressions and simple actions such as clapping and jumping [11, 12] Additionally, some studies integrate multimodal data, such as video, audio, electroencephalograms, and eye-tracking information, to extract basic facial and emotional features using deep learning models. These extracted features are then analyzed to facilitate the detection of ASD. [13], [14]. However, these studies have not employed large-scale multimodal self-supervised pretraining strategies. At the same time, the clinical application of recognizing only facial expressions and simple actions is limited. Implementing deep learning methods to automatically recognize behaviors within a comprehensive clinical schedule can play a highly beneficial role not only in diagnosing autistic patients but also in preventing and treating ASD.

### D. Multimodal Prompt Engineering

In recent years, especially following the release of ChatGPT, the advent of large AI models augmented with prompt engineering has presented a robust alternative approach to the conventional methods of constructing and training models for various AI tasks. These techniques have demonstrated exceptional performance across a range of professional fields, including medicine and law [15], [16]. Moreover, research has indicated its significant professional abilities in psychology and behavior recognition in individuals with autism [17], [18].

Regarding this study, we opted not to use GPT-4V fine-tuning as a comparative benchmark for our model primarily for three reasons. First, the vast number of parameters in GPT-4V entails considerable computational costs for training, which does not translate to clinical value. Second, due to its large parameter count, even inference computations on a fully trained model are challenging to deploy on local hospital systems due to constraints in VRAM or computational power. Third, as GPT-4V is a proprietary model of OpenAI, the company has not open-sourced the model weights, and the models available for online fine-tuning do not include the vision-language multimodal model GPT-4V.

Therefore, we decided to use the GPT-4V+Prompt Engineering method as our baseline for the FOS-II IS Encoding task.

A transformer-based audio-visual multi-modal interaction style recognition system is provided for children with autism based on the Family Observation Schedule (FOS-II). A deep-learning based algorithm with audio-visual multimodal-data clinically coded with the Family Observation Schedule 2nd Edition (FOS-II), named AV-FOS model. Our AV-FOS model leverages transformer based structure and self-supervised learning to intelligently recognize Interaction Styles (IS) in the FOS-II scale from subjects' video recordings. This enables the automatic generation of the FOS-II measures with clinically acceptable accuracy. We explore the IS recognition using a multimodal large language model, GPT4V, with prompt engineering provided with FOS-II measure definitions as the baseline for this study and compare with other vision-based deep learning algorithms. We believe this research represents a significant advancement in autism research and clinical accessibility. The AV-FOS and our FOS-II dataset will serve as a gateway toward the digital health era for future AI models related to autism.

### III. Methodology

In this section, we will discuss the dataset creation process, including data collection and labeling, as well as the construction and training of our AV-FOS model. It is noteworthy that our AV-FOS model utilizes a pre-training strategy, where the structure during pretraining differs from the structure used during formal training. The respective structures and training methods are discussed in Sections III.D and III.E.

### A. Dataset

1) Dataset Description: This dataset was designed to measure fine-grained FOS-II scales for detecting challenging behaviors in autistic children. Researchers recorded videos in participants' homes at the invitation of parents, providing realistic data to enhance clinical services such as ASD treatment, severity diagnosis, and symptom management. This real-life setting underscores the dataset's high clinical value.

The dataset comprises 216 videos, each 5 to 15 minutes long, from 83 participants. The videos were recorded at a frame rate of 30 frames per second, and the corresponding audio was captured at a sample rate of 16,000 Hz. Children with ASD were diagnosed by licensed clinicians, while those without a confirmed diagnosis met the ASD screening cutoff (≥15) on the Social Communication Questionnaire (SCQ). Participants had a mean age of 9.72 years (SD=4.77), with a male-to-female ratio of approximately 7:3.

Children performed daily tasks designed to assess cognitive, motor, and social skills. Current data focus on children aged 1 to 12, though tasks can be adapted for adolescents and adults in future studies. Problem behaviors ranged from mild to severe, evaluated using the Problem Behavior Checklist. This checklist measures 14 common behaviors (e.g., self-injury, aggression, repetitive movements, noncompliance, feeding issues, hyperactivity) on a 5-point Likert scale, with total scores ranging from 14 to 70. Higher scores indicate more frequent or severe behaviors, and participants in this study had a mean score of 33.00, reflecting moderate severity.

Handheld cameras were deliberately chosen to simulate uncontrolled environments, as this introduces a level of noise that enhances the model's robustness to real-world scenarios. While advanced IP-based cameras could provide higher resolution and stability, relying on handheld cameras ensures broader applicability by enabling future diagnostic systems to operate effectively without requiring complex and costly recording setups. Each video features one of three tasks: (1) playing with specific toys, (2) following a series of instructions (four versions available, as shown in Table I), or (3) free play.

TABLE I
THE DESIGN OF INSTRUCTION LISTS

| | Categories | Tasks |
|---|---|---|
| A | A. Gross motor control | Walks 10 steps by himself/herself |
| | B. Fine motor control | Leaves marks or draws on paper with pencil or crayon |
| | C. Social interaction | Follows instructions when asked to wave or clap his/her hands |
| | D. Language comprehension | Follows verbal instructions such as “put it over there” or “bring it over here” |
| | E. Language usage | Answers simple questions by shaking his/her hand or saying “yes/no” |
| | F. Table manner | Drinks water from the cup without spilling it |
| | G. Wearing clothes | Extends his/her limbs when changing clothes |
| | H. Personal Hygiene | Washes his/her hands by the sink with running water |
| | I. Mathematical ability | Counts from 1 to 5 |
| | J. Problem Solving | Chooses a particular tool/material out of many tools/materials |
| B | A. Gross motor control | Pours water from the cattle/jar into the cup |
| | B. Fine motor control | Closes the zipper on his/her clothes when wearing them |
| | C. Social interaction | Plays simple games (e.g., rolling balls) with other people |
| | D. Language comprehension | Identifies his/her name from a group of names that includes at least 4 other names |
| | E. Language usage | Names familiar objects such as cup, blanket, or ball |
| | F. Table manner | Eats food with fork |
| | G. Personal Care | Flushes the toilet after using it |
| | H. Wearing clothes | Wears shoes (shoes without laces) correctly |
| | I. Personal Hygiene | Uses handkerchief or tissue to blow and wipe his/her nose |
| | J. Household Chores | Disposes trash at appropriate places |
| C | A. Gross motor control | Catches bouncing ball (e.g., tennis ball) with two hands |
| | B. Fine motor control | Screws or places small components such as screws into the right place |
| | C. Social interaction | Searches or remembers his/her friends' phone number and calls them |
| | D. Language comprehension | Searches for the needed information from dictionary or encyclopedia |
| | E. Language usage | Writes his/her full name correctly with any assistance |
| | F. Table manner | Uses knife to cut the food into small pieces if it is too large to eat |
| | H. Personal Care | Ties shoelaces so they do not become untied |
| | I. Wearing clothes | Takes care of his/her nails (e.g., cutting, grinding) when needed |
| | J. Household Chores | Uses dustpan after sweeping the floor with a broom |
| | M. Problem solving | Asks an appropriate person for a tool or material when in need |
| D | A. Gross motor control | Does at least 6 push ups |
| | B. Fine motor control | Folds the letter into thirds, puts it into an envelope and seals the envelope with glue |
| | C. Social interaction | Plans to invite people into the house |
| | D. Language comprehension | Understands news articles or books after reading them |
| | E. Language usage | Summarizes news articles or books after reading them |
| | F. Table manner | Uses knife to cut the food into small pieces if it is too large to eat |
| | H. Wearing clothes | Wears innerwear first before wearing clothes |
| | I. Personal hygiene | Fixes his/her hair in front of the mirror |
| | J. Household Chores | Cleans with a vacuum cleaner |
| | M. Problem solving | Asks an appropriate person for a tool or material when in need |

2) Dataset Annotation: The videos in this dataset are annotated every 10 seconds using the FOS-II structured interval-based coding system to capture interaction styles (IS) between children and their caregivers, which serve as labels for training deep learning models. A total of 23 IS types are coded, encompassing both parental IS (e.g., Praise (P), Affection (AF)) and child IS (e.g., Non-compliance (NC), Opposition (O)).

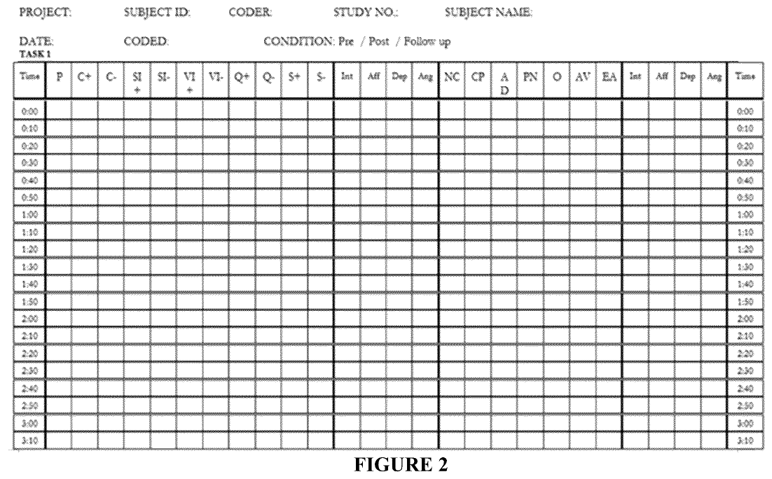

Some IS types are marked with positive or negative symbols to indicate emotional tone; for example, SA+ denotes positive social attention, while SA represents negative social attention. A detailed overview of IS codes is provided in Table II, and FIG. 1 illustrates several examples of IS annotations corresponding to video frames. If a behavior occurred during the interval, it was recorded as “1”. FIG. 2 shows the coding sheet used during the annotation process.

TABLE II
THE EXPLANATION OF EACH IS AND THE CORRESPONDING
FREQUENCY IN THE FOS-II DATASET

| IS Code | IS Name | Frequency |
|---|---|---|
| AD | Adhesive Demand | 41 |
| AV | Appropriate Verbal Interactions | 1464 |
| Aff child | Children Affection | 24 |
| Aff parent | Parent Affection | 329 |
| C+ | Positive Contact | 2223 |
| C− | Negative Contact | 15 |
| CP | Complaint | 178 |
| EA | Engaged Activity of Play | 3630 |
| Int child | Children Interrupt | 1 |
| Int parent | Parent Interrupt | 1 |
| MI | Multiple Instructions | 185 |
| NC | Non-compliance | 150 |
| O | Opposition | 2511 |
| P | Praise | 332 |
| PN | Physical Negative | 72 |
| Q+ | Positive Question | 1586 |
| Q− | Negative Question | 4 |
| S+ | Positive Social Attention | 5086 |
| S− | Negative Social Attention | 13 |
| SI+ | Positive Specific Instruction | 799 |
| SI− | Negative Specific Instruction | 13 |
| VI+ | Positive Vague Instruction | 2983 |
| VI− | Negative Vague Instruction | 20 |

The coding process was conducted manually by trained research assistants, who observed video recordings and documented whether a behavior occurred during each 10-second interval. Five trained graduate students from the Department of Psychology of Yonsei University served as human coders under the supervision of a licensed clinical psychologist with Board Certified Behavior Analyst (BCBA) credentials. Coders underwent extensive training, including 20 hours of practice and evaluation, to ensure annotation accuracy. They worked in pairs to establish inter-observer reliability, and inter-rater reliability was calculated on 30% of the dataset, yielding a 90% agreement rate, exceeding the acceptable threshold of 80%.

This rigorous annotation process ensures reliable labels for studying behavior patterns and training machine learning models.
3) Ethical Considerations and Privacy Implications: Informed consent was obtained in compliance with ethical guidelines through an explanatory document approved by the Institutional Review Board (IRB) of Yonsei University, where the study was conducted. Participants provided written consent for video recording, which was solely used for research purposes. The consent process included a detailed explanation of the study's objectives and the procedures for data protection and privacy. To ensure confidentiality, all video data were anonymized, with no personally identifiable information (e.g., names, dates of birth) included in the recordings. Each participant was assigned a unique numeric identifier to prevent identification. Access to video data was strictly limited to researchers who had received prior IRB approval. The data were securely stored in a password-protected, encrypted database to ensure robust security. Furthermore, we adhered to data retention and disposal policies as mandated by the IRB. All video data will be securely deleted following the completion of the study to uphold ethical standards. These procedures ensured the highest level of participant privacy and data integrity throughout the research process.

### B. Data Preprocessing (FIG. 4)

Referring to FIG. 4, for videos 100 originally ranging in length from 5 minutes to 15 minutes, we initially perform a trimming process to establish a dataset comprising clips 111 of 10 second duration each, annotated with corresponding Interaction Styles (10s FOS-II Dataset (though FOS-II is discussed herein, any suitable dataset can be utilized including FOS-III-R Dataset). Subsequently, we utilized the open-source Sound eXchange software and the OpenCV library to extract audio data 120 and video data from each 10-second video clip 111 for further processing.

FIG. 3 provides a visual comparison of three suitable approaches for visual information processing. The approaches sample and preprocess 10s video data, aiming to maximize the preservation of both spatial and temporal information. In all three approaches, the final output has 196 visual patches, which are input into the model for attention computation, feature extraction, and IS prediction:

$$v = [ {v}^{1} , {v}^{2} , … , {v}^{196} ] \qquad ( 1 )$$

Approach 1—Middle Frame Spatial Attention: We select the central frame of the video as the keyframe, resize it to 224×224 pixels, and divide it into 196 square patches.
Approach 2—Cross-Frame Attention: The video is divided into four temporal segments, and one keyframe is selected from each. These keyframes are resized to 112×112 pixels and divided into 49 square patches each, collectively forming 196 patches.
Approach 3—Averaged Key Frame Attention: We extract one keyframe each from the first, middle, and final thirds of the video, compute their pixel-level average image, resize it to 224×224 pixels, and divide it into 196 square patches.

The first approach prioritizes high-quality spatial information but includes minimal temporal information. The latter two approaches preserve more temporal information by slightly compromising spatial resolution. After evaluation, our Averaged Key Frame Attention demonstrated the best performance; thus, we selected this model for further analysis. Detailed results and discussion can be found in Section IV-F.3: Ablation Study Visual Temporal Information Perception.

For audio data 120 processing, the raw waveforms were first normalized by subtracting their mean value, centering the signals and ensuring consistent amplitude across all samples. The audio maintains its native sample rate (16000 HZ), preserving the original quality of the recordings. And then, Mel-filter bank (fbank) features were then extracted using a Hanning window with a window size of 25 ms and a frame shift of 10 ms. The extraction process generated 128-dimensional log Mel-filter bank features for each frame, resulting in a time-frequency representation of the audio data. To ensure uniform input dimensions for the model, the extracted spectrograms were adjusted to a fixed temporal length of 1024 frames through zero-padding for shorter spectrograms or trimming for longer ones.

Finally, the spectrograms were divided into 512 square patches of size 16×16, following a consistent representation format for input into the model. This pre-processing pipeline was designed to preserve critical temporal and spectral information, ensuring that the audio features were robust and aligned with the model architecture:

$$a = [ {a}^{1} , {a}^{2} , … , {a}^{512} ] \qquad ( 2 )$$

### C. Transformer-Based Encoder and Decoder

In both the pre-training (FIG. 5) and formal (FIG. 6) model structures, the Transformer-based Encoder and Decoder are integral components of our model. Therefore, this section introduces their internal structural details to facilitate the subsequent discussions on the pre-training and formal structures of the model in the following sections.
1) Tokenization: Initially, in the Tokenization phase, we embed not only positional information but also modality information, such as through the audio patch and video patch input layers. Specifically, for patch embedding, we use learnable linear projection (LP) layers 114, 124 to process the original square patch

$${p}_{m}^{i}$$

∈a∪v, where each modality m∈{audio, video} and i denotes the patch number. In the positional embedding

$$( {PE}_{m}^{i} ) ,$$

a fixed modality-specific 2-D sin-cos embedding strategy is employed. Modality embedding is accomplished using trainable parameters ω. Ultimately, by performing element-wise addition, we obtain the sequence of tokens input into the transformer block. Each token t in this sequence has a length, or embedding dimension, of 768. Consequently, the token

$${t}_{m}^{i}$$

can be mathematically expressed as:

$${t}_{m}^{i} = LP ( {p}_{m}^{i} ) + {PE}_{m}^{i} + {ω}_{m} \qquad ( 3 )$$

The whole process of data pre-processing and tokenization is shown in FIG. 4. In FIG. 4, the visual information (image data) 110 is processed by, for each 10s clip 111, first taking a Partial Average Image Calculation. The Averaged Key Frame Attention (FIG. 3) is the “Partial Average Image Calculation”+“Patch cutting” in FIG. 4. FIG. 4 includes Averaged Key Frame Attention algorithm but FIG. 3 shows more details about this structure. The operations of all the figures, here FIGS. 3, 4, can be implemented by the processing device (CPU/GPU) of FIG. 1A, which also provides a deep learning algorithm. The image data is then resized, center cropped, and normalized, then patches are cut 112. For pretraining, a mask is applied. A linear projection of the image data is applied to flatten the patches, and then position and modality encoding is used. For the audio information 120, for each 10s clip, a 128D log Mel filterbank and padding is applied, and patches are cut 122. For pertaining, a mask is applied. A linear projection of the audio data is then applied to flatten the patches, and then position and modality encoding is used.

The Encoders (210, 212, 220, 310, 312, 320) and Decoder (230) are composed of transformers, which makes the system transformer-based, thus providing the network with improved capacity to handle large amount of data with great learning capabilities. Transformer blocks: In each transformer block of the model, the architecture fundamentally adheres to the standard Transformer structure [19]. A transformer block includes a stack that follows a specific pattern of a Multi-Head Attention layer (MHA), residual connection layers, a Feed-Forward Neural Network/Multilayer Perceptron layer (MLP), and Layer Normalization layers (LN). For each input token sequence x=[t₁, t₂, . . . , tₙ] and the corresponding output token sequence y, the mathematical expressions are as follows:

$${x}^{′} = MHA ( {LN}_{1} ( x ) ) + x \qquad ( 4 )$$

$$y = MLP ( {LN}_{2} ( {x}^{′} ) ) + {x}^{′}$$

Here, LN₁ and LN₂ represent the layer normalization steps applied before the multi-head attention and feed-forward neural network.
3) Encoder and Decoder: The encoder Em(·) structures 210, 212, 220, 310, 312, 320 and decoder Dₘ(·) structures 230 are similar to those in the MAE [20] but accept different modality tokens. The encoder is a sequence of transformer blocks applied only to visible, unmasked tokens. Conversely, the decoder is also composed of a sequence of transformer blocks; however, the input to the decoder comprises the full set of tokens, including both masked and unmasked tokens. Each masked token is a shared, learned vector that indicates the presence of a missing patch to be predicted, and both positional embeddings and modality embeddings are added to the tokens. For the different modality encoder and decoder, the structure is the same. We assume that this consistent structure will enhance the performance of modality fusion perception for the multimodal task.

### D. Self-Supervised Model Pretraining (FIG. 5)

Referring now to FIG. 5, the present system is shown utilizing a pretraining system 200, leveraging relatively low-cost unlabeled data for prior knowledge acquisition, thereby enabling the use of more data for training in future research, which holds greater potential. We adhere to the original CAV-MAE algorithm for our model initialization and pretraining, as depicted in FIG. 5.

As shown, the pretraining system 200 includes a pretraining input 202, 204, pretraining audio encoder 210, pretraining video encoder 212, a pretraining joint encoder 220, and a pretraining joint decoder 230. The pretraining input can be, for example, audio patches 202 and video patches 204. A mask 206 is applied to the input data 202, 204 to obtain masked audio data A1-An and masked video data V1-Vn 208. The system uses reconstruction loss to train the model and test the reconstruction result to train the model. The masked part of video or audio can serve as a label or ground truth in our self-supervised learning structure. In some embodiments, the mask randomly removes data from the input data 202, 204, and here for example A2, A4, V2, V4 are masked out.

The pretraining audio encoder 210 receives the masked audio patches A1 . . . An from the input and generates encoded audio embedding E_{A1}-E_{An}. Notably, because the audio data A2 was removed by the mask, there is no encoded audio embedding E_{A2}. The pretraining video encoder 212 receives the masked video patches V1 . . . . Vn from the input and generates encoded video embedding E_{V1}-E_{Vn}. Because the video data V2 was removed by the mask, there is no encoded audio embedding E_{V2}. The system then duplicates the audio embedding and video embedding into two copies. One pair 214, 216 is fed into the pretraining joint encoder 220 to obtain further embeddings 224 and 226, which are used for the model's contrastive learning. The other pair of audio and video embeddings is first concatenated to form a fused embedding 215, which is also fed into the pretraining joint encoder 220 to generate a fused embedding 225. This fused embedding 225 is used for the model's reconstruction learning.

Subsequently, the audio embedding 224 undergoes a pooling operation to obtain C_{A1} and the visual embedding 226 is also pooled to obtain C_{V1}. The system uses C_{A1} and C_{V1} to compute the contrastive loss 227 for contrastive learning. Meanwhile, the aggregated embedding 225 is first supplemented with a predefined mask token at the masked positions to produce embedding 228. This embedding 228 is then fed into the pretraining joint decoder 230 to reconstruct both audio and visual information. By comparing the reconstructed outputs with the original masked data, we compute the reconstruction loss 232, which is used for reconstruction learning. As noted, the operation of FIG. 5 can be implemented by the processing device 16 (FIG. 1).

The masking and the training process to learn to fill in the masked patches along with clinical measure embeddings gives great advantages in learning to distinguish the clinical measures in the training data given only 10 seconds of input data, making the network learn to guess the scene even from the limited amount of data and estimating the clinical measures as well.

Turning to FIG. 6, the FOS-II decision neural network audio-visual transformer-based system (AV-FOS) 300 (e.g., for supervised learning, discussed in section E below) in accordance with one example embodiment of the disclosure. As shown, the AV-FOS system 300 includes an AV-FOS input, AV-FOS audio encoder 310, AV-FOS video encoder 312, and an AV-FOS joint encoder 220. The AV-FOS input can be, for example, AV-FOS audio patches 302 and AV-FOS video patches 304. The AV-FOS audio encoder 310 receives the audio patches A1 . . . . An from the input and generates encoded audio data E_{A1}-E_{An}. The AV-FOS video encoder 312 receives the masked video patches V1 . . . Vn from the input and generates encoded video data E_{V1}-E_{Vn}. The encoded audio data E_{A1}-E_{An} and the encoded video data E_{V1}-E_{Vn} are then embedded and encoded to form concatenated AV-FOS audio/video data E_{V1}-E_{Vn}, E_{A1}-E_{An} 215. The input structure (which is the same (pre-processed and tokenized by using the structure of FIG. 4) for both training (FIG. 5) and supervised training (FIG. 6), though for training data a mask is applied.

The audio and video input patches 302, 304 are not masked (as in FIG. 5), and the encoded audio data E_{A1}-E_{An} and encoded video data E_{V1}-E_{Vn} are not separately embedded (as in FIG. 5). In this training stage, the system does not do the self-supervised pre-train (not use reconstruction learning and contrastive learning), so the structure for the IS recognition task and classification supervised learning is not applied.

The AV-FOS joint encoder 320 receives and jointly encodes the embedded concatenated encoded audio/video embedding E_{A1}-E_{An}, E_{V1}-E_{Vn} 315 to generate encoded concatenated embedding E_{A1}-E_{An}, E_{V1}-E_{Vn} 325. Then, the concatenated embedding 325 undergoes a token-level mean pooling operation 340 to produce a feature vector, which is fed into the joint IS decision-making Multilayer Perceptron (MLP) layers 342 to generate the final feature vector 344. Each value in this feature vector represents a specific Interaction Style (IS); if the value exceeds the predefined threshold of 0.4, the model determines that the corresponding IS is present in the video. The one-hot vector 346 represents the human-annotated labels and is used to compare with the model's prediction during training. It is not required during inference. All computers used for model development in this work were Lambda 2 servers, each equipped with four Nvidia A5000 GPUs.
1) Loss Function: Generally, our approach aims to leverage the inherent connections within 1) video information and its corresponding audio information, and 2) patches within the same contextual data. Consequently, we employ both Contrastive Loss and Reconstruction Loss as our loss functions. The reduction in contrastive Loss indicates that visual and audio information from the same context are brought closer in the feature space, while data from different contexts are distanced. On the other hand, reconstruction Loss is calculated by initially masking most patches and then generating the masked data using a limited set of features in the feature space along with a Transformer decoder 230 (FIG. 5). The loss is then assessed based on the difference between the generated data and the original data. A decrease in Reconstruction Loss indicates that the model has learned the latent connections between contextual data. The computation and application of these two types of losses do not rely on manual annotations, which substantially reduces labeling costs and enhances the model ability to extract features from input data. This is advantageous for our model performance on the self-collected FOS-II dataset.
2) Model Structure: The input tokens from the two modalities are initially subjected to a masking process, which obscures 75% of the tokens. Subsequently, these masked tokens are fed into their respective modality-specific encoders, resulting in the preliminary embedding outcomes 214, 215, 216, denoted as ^{e}^{ⁱ}_{unmask_a} and ^{e}^{ⁱ}_{unmask_v}ⁱ.

$${e}_{unmask \_ a}^{i} = {Mask}_{0.75} ( {E}_{a} ( {t}_{a}^{i} ) ) \qquad ( 5 )$$

$${e}_{unmask \_ v}^{i} = {Mask}_{0.75} ( {E}_{v} ( {t}_{v}^{i} ) )$$

For clarity, it is noted that the variables in the figures differ from those here. The variables in the figures are simplified for easier understanding, for example in FIG. 5 the variables are indicated as are EA1, EV1, etc. After passing through the initial unimodality encoders 210, 212, the two modality embeddings ^{e}^{ⁱ}_{unmask_a} and ^{e}^{ⁱ}_{unmask_v}ⁱ are directly input into the Joint Encoder Eⱼ(·) 220 where a Mean Pool operation is conducted to obtain

$${c}_{a}^{i} and {c}_{v}^{i}$$

for computing the contrastive loss 227. Here, Bᵢ denotes the i-th video clip from the current training batch B. Simultaneously, in order to calculate the reconstruction loss, these two vectors are concatenated and then fed into the Joint Encoder 220, resulting in the aggregated embeddings sequence e_{unmask_m} 225 which is prepared for subsequent reconstruction operations.

$${c}_{a}^{i} = MeanPool ( {E}_{j} ( {e}_{unmask \_ a}^{i} ) ) \qquad ( 6 )$$

$${c}_{v}^{i} = MeanPool ( {E}_{j} ( {e}_{unmask \_ v}^{i} ) )$$

$${e}_{unmask \_ m} = {E}_{j} ( [ {e}_{unmask \_ a} , {e}_{unmask \_ v} ] ) \qquad ( 7 )$$

The computation of the contrastive loss [symbol] _{c} is as follows:

$${ℒ}_{c} = - \frac{1}{N} {\sum }_{i = 1}^{N} log ( \frac{exp ( {s}^{i , i} / τ )}{{\sum }_{k \neq i} exp ( {s}^{i , k} / τ ) + exp ( {s}^{i , i} / τ )} ) \qquad ( 8 )$$

Where

$${s}^{i , j} = {‖ {c}_{v}^{i} ‖}^{T} ‖ {c}_{a}^{j} ‖$$

and [symbol] is the temperature.

For the reconstruction loss calculation 232, we pad e_{unmask_m} at the original masked position as eₘ and elementwise add the fixed sinusoidal positional and learnable modality embedding (PEₘⁱ and ωₘ). And then pass the decoder structure to get the reconstruction of the original audio and video patch âᵢ and \hat{v}ᵢ.

$${\hat{a}}^{i} = {D}_{j} ( {e}_{a}^{i} + {PE}_{a}^{i} + {ω}_{a} ) \qquad ( 9 )$$

$${\hat{v}}^{i} = {D}_{j} ( {e}_{v}^{i} + {PE}_{v}^{i} + {ω}_{v} )$$

We then apply a mean square error reconstruction loss [symbol] ᵣ.

$${ℒ}_{r} = - \frac{1}{N} {\sum }_{i = 1}^{N} [ \frac{\sum {( ( {\hat{a}}_{mask}^{i} ) - norm ( {a}_{mask}^{i} ) )}^{2}}{\vert {a}_{mask} \vert} + \frac{\sum {( ( {\hat{v}}_{mask}^{i} ) - norm ( {v}_{mask}^{i} ) )}^{2}}{\vert {v}_{mask} \vert} ] \qquad ( 10 )$$

Here, N denotes the mini-batch size, and |aⁱₘₐₛₖ| and |vⁱₘₐₛₖ| denote the number of masked audio and visual patches, respectively.

Finally, we sum the contrastive loss [symbol] _{c} and reconstruction loss [symbol] ᵣ as the final loss [symbol] :

$$ℒ = {λ}_{c} {ℒ}_{c} + {ℒ}_{r} \qquad ( 11 )$$

Here, λ_{c}∈[0, 1] represents the ratio of the contrastive loss.
3) Model Initialization and Pretrained dataset: In this study, we utilized the pretrained model weights from the CAV-MAE paper, which were used to initialize our model, specifically CAV-MAE^{scale+}. These weights were obtained through pretraining on the AudioSet dataset [21].

### E. FOS-II Encoding Model Supervised Learning

To facilitate the model ability to learn more prior knowledge conveniently, during the pretraining phase, we incorporated numerous redundant structures such as decoders and patch masking. However, before proceeding with supervised training on the self-collected FOS-II dataset, it is necessary to modify the model structure. This involves removing redundant components while retaining the neural network layers that store the most prior knowledge. Additionally, we introduce appropriate classification layers and employ different loss functions to train the model, optimizing it for the multi-label classification task of FOS-II Interaction Styles (IS). This newly constructed and trained network is named the Audio-Visual FOS-II Encoding Neural Network (AV-FOS), which is specifically designed for recognizing FOS-II IS in the medical domain.

The preprocessing and tokenization of input data for AV-FOS remain consistent with previous discussions, except for the elimination of the masking step. The input audio patches a=[a¹, a², . . . , a⁵¹²] and video patches v=[v¹, v², . . . , v¹⁹⁶] undergo tokenization and element-wise addition of positional and modality embeddings, resulting in tₐ=[t¹, t², . . . , t⁵¹²] and tᵥ=[t¹, t², . . . , t¹⁹⁶], respectively. These tokens are then input into their respective modality-specific encoders, which have been pretrained, followed by concatenation and input into a previously pretrained Joint Encoder to obtain the feature vector

$${e}_{m} = [ {e}_{a}^{1} , {e}_{a}^{2} , … , {e}_{a}^{512} , {e}_{v}^{1} , {e}_{v}^{2} , … , {e}_{v}^{196} ] :$$

$${e}_{m} = {E}_{i}^{*} [ {E}_{a}^{*} ( {t}_{a} ) , {E}_{v}^{*} ( {t}_{v} ) ] \qquad ( 12 )$$

Here, the asterisk (*) indicates that the module has undergone pretraining.

We employed a token level mean pooling strategy: for each embedding dimension (out of 768), we compute the average across all tokens to generate an average token. This average token (a vector of length 768) serves as a mapping of all real-world information in the feature space, which is highly suitable for FOS-II classification. This vector is then input into MLP of the decision layer, denoted ISMLP(·), to produce a feature vector vIS of length equal to the number of labels (FOS-II IS), which is 13:

$${v}_{IS} = ISMLP ( Mean ( {e}_{m} ) ) \qquad ( 13 )$$

Subsequently, if performing inference, this vector is processed through a Sigmoid function, compared with a manually defined threshold θ, and if it exceeds this threshold, the IS is determined to be present in the input 10-second video:

$${IS}_{detected} = \{ i \vert Sigmoid ( {v}_{IS}^{i} ) > θ \} \qquad ( 14 )$$

During the training process, the output of the model vis is first processed through a Sigmoid function, and then the Binary Cross-Entropy (BCE) Loss [symbol] _{BCE} (FIG. 6) is computed with respect to the ground truth 346 one-hot encoded vector vGT. This loss is then used to guide the training of the model:

$${p}_{IS} = Sigmoid ( {v}_{IS} ) \qquad ( 15 )$$

$${ℒ}_{BCE} = - {\sum }_{i = 1}^{N} ( {v}_{GT}^{i} \cdot log {p}_{IS}^{i} + ( 1 - {v}_{GT}^{i} ) \cdot log ( 1 - {p}_{IS}^{i} ) )$$

The currently trained AV-FOS model exhibit 164.512 million parameters.

F. GPT4V Prompt Engineering with FOS-II Definitions

We have employed OpenAI state-of-the-art multimodal foundation model, GPT-4V, [5] combined with prompt engineering as the baseline for our FOS-II IS Encoding task.
1) Prompt Engineering: We designed two versions of prompts (Prompts V1 & V2), each having two components: a textual prompt and a visual information prompt. The first version of the visual information prompt (Prompt V1) includes the starting, middle, and ending frames from the original 10-second video. The textual prompt guides the model to utilize this three-frame information to facilitate the GPT-4V in recognizing FOS-II IS. The design of the textual component of the first version of the prompt is as follows:
A video is given by providing three frames in chronological order. Please choose one or more appropriate interaction styles or behaviors in the video. Please only reply with the numbers of the interaction styles or behaviors, separated by commas. The candidates of the interaction styles or behaviors are as follows: 1. Appropriate verbal interactions 2. Parent affection 3. Positive contact 4. Complaint 5. Engaged activity of play 6. Multiple instruction 7. Non-compliance 8. Oppositional 9. Praise 10. Positive question 11. Positive social attention 12. Positive specific instruction 13. Positive vague instruction.

The second version of the prompt (Prompt V2) incorporates a brief explanation of each interaction style within the textual part, while the video component utilizes a method of randomly selecting three key frames. These key frames are extracted randomly from the first third, middle third, and final third of the original 10-second video. The design of the textual prompt for the second version is as follows:
A video is given by providing three frames in chronological order. Please choose one or more appropriate interaction styles or behaviors in the video. Please only reply with the numbers of the interaction styles or behaviors, separated by commas. The candidates of the interaction styles or behaviors are as follows: 1. Appropriate verbal interactions: Appropriate verbal interactions are scored when a child engages in non-aversive, intelligible speech directed at others or self. 2. Parent affection: Parent affection is verbal or non-verbal affection, including words, physical contact, and leveling actions towards the child. 3. Positive contact: Positive contact is friendly, affectionate, or neutral physical interaction initiated or maintained by the parent. 4. Complaint: A complaint is any instance of whining, crying, or other vocal protests displaying temper or discontent. 5. Engaged activity of play: Engaged activity of play is scored when a child quietly plays, observes, or eats without deviance for a full interval. 6. Multiple instruction: Multiple instruction is scored when a parent gives more than one command or request in a single utterance. 7. Non-compliance: Non-compliance occurs when a child fails to follow a given instruction within five seconds or immediately contradicts it verbally. 8. Oppositional: Oppositional behavior is socially inappropriate or unacceptable child behavior not following specific family rules. 9. Praise: This category scores praise for specific behaviors or characteristics of the child, positively and non-aversively. 10. Positive question: A positive question is a non-aversive, information-seeking utterance from the parent to the child. 11. Positive social attention: Positive social attention is non-aversive verbal or non-verbal engagement by the parent that doesn't fit other categories. 12. Positive specific instruction: A Positive specific instruction is a direct, clear command with a defined behavioral expectation, delivered non-aversively. 13. Positive vague instruction: A Positive vague instruction is an indirect, non-aversive command without a clear behavioral referent.
2) Data Privacy with GPT4V API: Initially, we consulted OpenAI official website [22] concerning the privacy of API users, which guarantees that all data accessed via the API will be deleted within 30 days and further assures that such data will not be used to train future AI models. Additionally, the data we uploaded to the OpenAI API contained no personally identifiable information such as names, ages, or nationalities, nor did it indicate that the images originated from autistic children. Therefore, the use of the API for FOS-II IS recognition complies with the privacy protection regulations concerning human subjects outlined in the IRB protocol.

### IV. RESULTS AND EVALUATIONS

This section presents the experimental setup, the processing of the FOS-II dataset, the construction details of the AV-FOS model, and a performance comparison between the AV-FOS model and the GPT4V+Prompt Engineering (baseline) method. Additionally, we compare our model with other mainstream video recognition models based on CNN and Transformer architectures. Ablation experiments were also conducted to investigate the performance of various submodules and their impact on the overall AV-FOS model.

TABLE III
THE KEY HYPERPARAMETERS FOR
AV-FOS MODEL TRAINING STAGE

| Training stage | Pre-Training | Formal Training |
|---|---|---|
| Epochs | 25 | 100 |
| Batch size | 4 × 27 | 128 |
| Initial Backbone LR | 2e−4 | 1e−5 |
| Initial Classification layers LR | — | 2e−6 |
| LR decay start epoch | 10 | 5 |
| LR decay rate | 0.5 | 0.95 |
| LR decay step | 5 | 1 |

| | |
|---|---|
| Optimizer | Adam |
| | weight decay = 5e−7 |
| | betas = (0.95, 0.999) |

### A. Example Setup

1) Experiment Device: All experiments in this paper, including the training and inference of all deep learning models, were conducted on a server with four NVIDIA A5000 GPUS (Lambda-quad 2). Compared to enterprise-grade servers, this server is not only cost-effective but also moderately sized, akin to a typical household computer, making it highly suitable for deployment in hospital settings.
2) Training Details: During the pre-training and formal training stages, the Encoder part of this model has a total of 12 transformer blocks. The single-modality Encoder layer contains 11 transformer blocks, while the joint Encoder comprises only one transformer block. The model Decoder part includes eight transformer blocks. The Transformer blocks in the Encoder have 12 attention heads and an embedding dimension of 768. In contrast, the Transformer blocks in the Decoder have 16 attention heads and an embedding dimension of 512.

For calculating the contrastive Loss, the temperature τ is set to 0.05, while for computing the CAV-MAE Loss, λc is set to 0.01 and the IS decision threshold θ is set to 0.4. The remaining key hyperparameters for both the pre-training and formal training stages on the FOS-II dataset are shown in the table III.

### B. Dataset Segmentation

We processed the original dataset by slicing it into 8,108 video clips, each 10 seconds long, with corresponding IS annotations. The frequency of each IS annotation within the 8,108 clips is shown in Table II. Due to the low occurrence of certain IS annotations (fewer than 100 instances), which makes them unsuitable for deep learning training, we discarded these IS annotations. While this exclusion limits the model's ability to generate a complete Functional Observation Scale (FOS), thereby impacting its immediate clinical applicability, many of the discarded categories—such as Int parent (Parent Interrupt: instances where parents interact with others rather than the target children)—are of limited clinical significance. Furthermore, we consider data collection to be an ongoing effort. As the dataset expands in the future, retraining the model using the architecture can address this limitation. After dropping the data without annotations, we obtained a dataset containing 8,040 video clips, each 10 seconds long, with 13 types of IS annotations for training and validation.

To simulate the clinical environment as closely as possible during dataset splitting and to evaluate the model ability to generalize to previously unseen subjects, we adopted a subject-based data partitioning strategy. Data from 11 subjects were extracted as the validation set, having 1,867 10-second video clips, while the remaining subjects data were used as the training set, comprising 6,173 10-second video clips. Due to differences in behavioral patterns across subjects, the overall label (IS) distribution differs significantly between the training and validation sets, posing a considerable challenge to our model. The Table IV summarizes the occurrence of IS labels in the training and validation sets.

TABLE IV
LABEL DISTRIBUTION IN TRAINING
AND VALIDATION SETS

| | |
|---|---|
| Training | Validation |

| Label | Count | Proportion | Count | proportion |
|---|---|---|---|---|
| C+ | 1827 | 10.91% | 396 | 8.41% |
| Q+ | 1210 | 7.23% | 376 | 7.98% |
| S+ | 3821 | 22.82% | 1265 | 26.86% |
| AV | 883 | 5.27% | 581 | 12.34% |
| EA | 2880 | 17.20% | 750 | 15.92% |
| SI+ | 672 | 4.01% | 127 | 2.70% |
| VI+ | 2387 | 14.25% | 596 | 12.65% |
| O | 2053 | 12.26% | 458 | 9.72% |
| NC | 124 | 0.74% | 26 | 0.55% |
| P | 287 | 1.71% | 45 | 0.96% |
| AFF | 288 | 1.72% | 41 | 0.87% |
| parent | | | | |
| MI | 161 | 0.96% | 24 | 0.51% |
| CP | 153 | 0.91% | 25 | 0.53% |

### C. Metrics for Evaluating Model Performance

In this study, since it is a multi-label task, we evaluated the model using several metrics, including Accuracy, F1 Score, Strict Accuracy, AUC (Area Under the ROC Curve), and mAP. The formulas for these metrics are as follows:

$$Accuracy = \frac{1}{N} {\sum }_{i = 1}^{N} \frac{\vert {Y}_{i} ⋂ {\hat{Y}}_{i} \vert}{\vert {Y}_{i} ⋃ {\hat{Y}}_{i} \vert} \qquad ( 16 )$$

$$F 1 Score = \frac{2 \cdot Precision \cdot Recall}{Precision + Recall} \qquad ( 17 )$$

$$where :$$

$$Precision = \frac{{\sum }_{i = 1}^{N} \vert {Y}_{i} ⋂ {\hat{Y}}_{i} \vert}{{\sum }_{i = 1}^{N} \vert {\hat{Y}}_{i} \vert} \qquad ( 18 )$$

$$Recall = \frac{{\sum }_{i = 1}^{N} \vert {Y}_{i} ⋂ {\hat{Y}}_{i} \vert}{{\sum }_{i = 1}^{N} \vert {Y}_{i} \vert}$$

$$Strict Accuracy = \frac{1}{N} {\sum }_{i = 1}^{N} 𝕀 ( {Y}_{i} = {\hat{Y}}_{i} ) \qquad ( 19 )$$

where [symbol] is the indicator function that returns 1 if the argument is true and 0 otherwise.

$$AUC = \frac{1}{\vert 𝒴 \vert} {\sum }_{k ∈ 𝒴} {AUC}_{k} \qquad ( 20 )$$

where AUCₖ is the AUC for the k-th label.

$$mAP = \frac{1}{\vert 𝒴 \vert} {\sum }_{k ∈ 𝒴} {AP}_{k} \qquad ( 21 )$$

where APₖ is the Average Precision for the k-th label. These evaluation metrics reflect not only the absolute performance of the model but also its ability to handle imbalanced datasets.

### D. GPT-4V Result Post-Processing

GPT-4V generates three types of outputs: ideal outputs, problematic outputs, and unsolvable outputs. Ideal outputs follow the structure specified in the prompt, returning several numerical indices separated by commas. These outputs can be processed with a simple string-splitting algorithm. Problematic outputs return predicted IS but not in the format specified in the prompt, including both numerical indices and IS names. For these cases, we use code to extract the numerical indices. Unsolvable outputs occur when GPT-4V returns a descriptive statement indicating its inability to process the data. In such cases, the data is manually classified as having no IS present. Table V presents examples of the three different types of outputs.

TABLE V
THE THREE TYPES OF OUTPUTS FOR THE GPT-4V MODEL

| | | Occurrences | Occurrences |
|---|---|---|---|
| Output | Example | (Prompt V1) | (Prompt V2) |
| Ideal | 2. 3. 5. 11 | 1863 | 1843 |
| Output | | | |
| Problematic | 5. Engaged activity of | 3 | 7 |
| Output | play | | |
| Unsolvable | The images are too dark | 1 | 15 |
| Output | to accurately discern any | | |
| | specific interaction styles | | |
| | or behaviors. | | |

### E. Model Performance

In our experiments, we used GPT-4V with prompt engineering as the baseline, and also tested two classic models for comparison: the advanced video understanding model SlowFast Networks [6] based on the CNN structure, and the classical visual understanding model vision Transformer (ViT) [7] based on the Transformer structure. Both models were pretrained using supervised learning on large-scale public datasets as mentioned in their original papers. For SlowFast Networks, we selected the R50 architecture, pretrained on the Kinetics-400 dataset and fine-tuned on our FOS-II dataset. For ViT, we selected the ViT-base architecture with a patch size of 16×16 for input tokens, pretrained on the ImageNet-21k [24] and ImageNet 2012 datasets, and fine-tuned on our FOS-II dataset.

TABLE VI
THE GLOBAL PERFORMANCE COMPARISON OF DIFFERENT MODELS

| | | Fl | Strict | | | Time Cost |
|---|---|---|---|---|---|---|
| | Accuracy | Score | Accuracy- | AUC | mAP | (Per Sample) |
| GPT4V − prompt V1 | 0 7965 | 0.4581 | 0.1355 | 0.6624 | 03181 | 43349 ± 2.5927 |
| GPT4V + Promp < V2 | 07668 | 03330 | 0 1468 | 0.5896 | 0.2481 | 3.9792 ± 1.1968 |
| Slow-Fast | 0.8287 | 0.5437 | 0.1125 | 0.8445 | 0.6138 | 0.0031 ± 0.0088 |
| ViT | 0.8172 | 0 5448 | 0.0889 | 0.8486 | 0.6167 | 0.0011 ± 0.0022 |
| AV-FOS (Our | 0.8590 | 0.5936 | 0.2003 | 0.8868 | 0.6879 | 0.0018 ± 0.0003 |
| Proposed) | | | | | | |

1) General Performance: Table VI and FIG. 7 present a comparison of various performance metrics across different models. The results show that our model significantly outperformed the baseline GPT-4V model not only in terms of accuracy and the ability to handle imbalanced datasets but also in inference speed. Additionally, our model performance exceeded that of the comparison models, SlowFast Networks and ViT. When tested on subjects that the model had never encountered before, our model still achieved an accuracy of over 85%, demonstrating robust performance. This surpasses the 80% inter-rater reliability standard, though it falls slightly short of the 90% agreement level achieved by human annotators in this study. However, our model has the potential to be further optimized through the continuous collection of new data. Additionally, when faced with an extremely imbalanced dataset, our AUC, mAP, and F1 scores reached 0.88, 0.67, and 0.59, respectively, indicating that the model demonstrates a significant advantage in handling imbalanced datasets. In terms of inference time, the baseline GPT-4V model lags significantly behind our model. For a 10-second video, our model requires only an average of 0.0018 seconds to complete inference, achieving real-time inference speed. These metrics indicate that our model has high clinical value and can assist doctors and healthcare providers in the diagnosis and risk behavior assessment for autistic children.

TABLE VII
DETAILED COMPARISON OF MODEL PERFORMANCE ACROSS CLASSES

| | AV-FOS (PtcpOKd) | | | SlowFast | | | ViT | | |
|---|---|---|---|---|---|---|---|---|---|
| | Accuracy | AUC | AP | Accuracy | AUC | AP | Accuracy | ACC | AP |
| AV | 0.7156 | 0.8383 | 0.6346 | 0.6920 | 0.6323 | 0.4237 | 0.6883 | 0.6080 | 0.4072 |
| Aff | 0.9780 | 0.8178 | 0.1038 | 0.9780 | 0.6198 | 0.0476 | 0.9775 | 0.6859 | 0.0577 |
| C+ | 0.7750 | 0.7559 | 0.4368 | 0.7675 | 0.8003 | 0.5477 | 0.7542 | 0.7795 | 0.4454 |
| CP | 0.9861 | 0.7010 | 0.0423 | 0.9866 | 0.5627 | 0.0210 | 0.9861 | 0.5420 | 0.0171 |
| EA | 0.6974 | 0.7624 | 0.6976 | 0.5008 | 0.6010 | 0.4959 | 0.4237 | 0.5577 | 0.4582 |
| Ml | 0.9877 | 0.8273 | 0.1050 | 0.9871 | 0.6422 | 0.0211 | 0.9871 | 0.7767 | 0.0378 |
| NC | 0.9861 | 0.8154 | 0.0760 | 0.9861 | 0.5969 | 0.0258 | 0.9861 | 0.6509 | 0.0499 |
| O | 0.7788 | 0.8012 | 0.5164 | 0.7392 | 0.6214 | 0.3557 | 0.6818 | 0.6831 | 0.3520 |
| P | 0.9759 | 0.7910 | 0.0747 | 0.9625 | 0.6263 | 0.0674 | 0.9754 | 0.6945 | 0.0588 |
| Q+ | 0.7986 | 0.7484 | 0.3409 | 0.7970 | 0.6393 | 0.2888 | 0.7991 | 0.6732 | 0.3368 |
| S+ | 0.3866 | 0.9071 | 0.9444 | 0.7761 | 0.8363 | 0.9090 | 0.8024 | 0.8734 | 0.9376 |
| SI+ | 0.9079 | 0.7606 | 0.2015 | 0.9272 | 0.6404 | 0.1121 | 0.9320 | 0.6543 | 0.1248 |
| VI+ | 0.7429 | 0.8205 | 0.6604 | 0.6733 | 0.7218 | 0.4925 | 0.6304 | 0.6882 | 0.4386 |

| | GPT4V + Prompt VI | | | GPT4V + Prompt V2 | | |
|---|---|---|---|---|---|---|
| | Accuracy | AUC | AP | Accuracy | AUC | AP |
| AV | 0.7070 | 0.6420 | 0.4155 | 0.7070 | 0.5462 | 0.3533 |
| Aff | 0.7745 | 0.5867 | 0.0285 | 0.5410 | 0.6342 | 0.0310 |
| C+ | 0.7418 | 0.7716 | 0.4011 | 0.6808 | 0.7762 | 0.3837 |
| CP | 0.9759 | 0.5143 | 0.0147 | 0.9636 | 0.5081 | 0.0137 |
| EA | 0.4660 | 0.5375 | 04207 | 0.5217 | 0.5672 | 0.4376 |
| Ml | 0.9813 | 0.4970 | 0.0129 | 0.9850 | 0.4989 | 0.0129 |
| NC | 0.9630 | 0.5073 | 0.0142 | 0.9748 | 0.4943 | 0.0139 |
| O | 0.7525 | 0.5001 | 0.2453 | 0.7542 | 0.4996 | 02453 |
| P | 0.9716 | 0.5195 | 0.0304 | 0.9555 | 0.4896 | 0.0241 |
| Q+ | 0.7981 | 0.5007 | 0.2017 | 0.7949 | 0.5136 | 0.2103 |
| S+ | 0.6631 | 0.6874 | 0.7793 | 0.4879 | 0.6173 | 0.7507 |
| SI+ | 0 8800 | 0.4940 | 0.0674 | 0.9207 | 0.5086 | 0.0702 |
| VI+ | 0.6792 | 0 4993 | 0.3190 | 0.6808 | 0.5000 | 03192 |

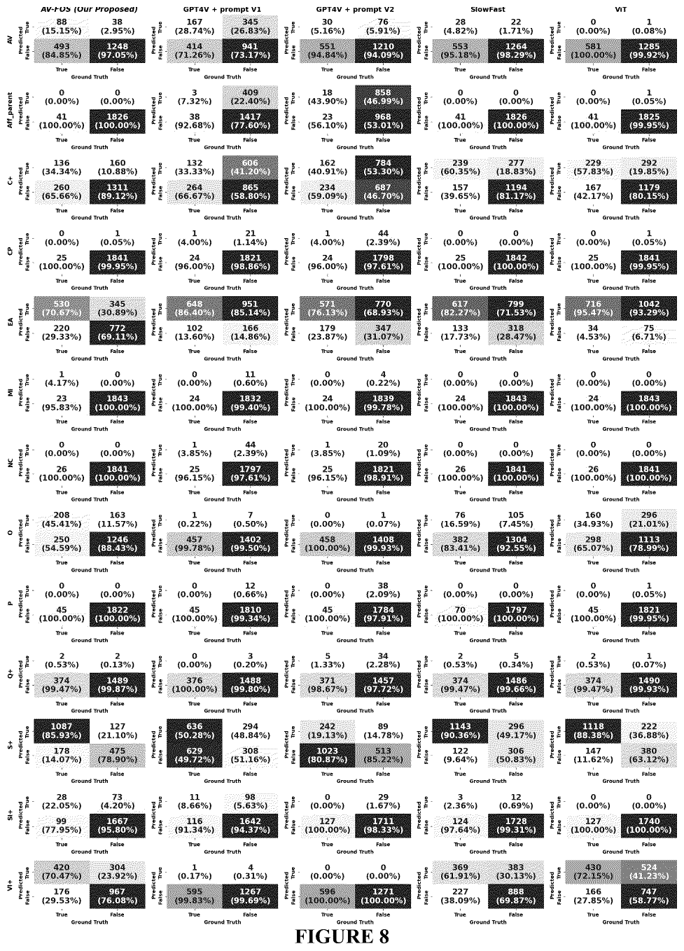

2) Class-Wise Evaluation and Error Analysis: The class-wise metrics presented in Table VII and the confusion matrix in FIG. 8 highlight the ability of different models to recognize various categories of interaction styles (IS). Compared to the baseline method, GPT-4V with prompt engineering, our model demonstrates significantly stronger recognition capabilities across all categories, consistently surpassing the baseline performance. Furthermore, unlike traditional video recognition models such as ViT and SlowFast Networks, our model incorporates the ability to process audio within videos. This capability gives our model a distinct advantage in recognizing IS that require audio comprehension, such as Positive Vague Instruction (VI+) and Positive Specific Instruction (SI+).

Interestingly, for IS typically associated with audio comprehension, other visual-only models also show some recognition ability. This can be attributed to the presence of visual cues during conversations, such as head turns and lip movements, which aid these models in making predictions. However, even for IS primarily reliant on visual information, such as Engaged Activity of Play (EA), our model maintains an edge over other models, demonstrating superior overall performance.

TABLE VII
WILCOXON SIGNED-RANK TEST RESULTS
BETWEEN AV-FOS AND COMPETING MODELS

| | | | | | GPT4V + | | GPT4V + | |
|---|---|---|---|---|---|---|---|---|
| | SlowFast | | ViT | | Prompt VI | | Prompt V2 | |
| Metric | W | p-value | W | p-value | W | p-value | W | p-value |
| Accuracy | 7.0 | 0.0208 | 9.0 | 0.0328 | 0.0 | 0.0002 | 5.0 | 0.0024 |
| AUC | 1.0 | 0.0005 | 1.0 | 0.0005 | 1.0 | 0.0005 | 1.0 | 0.0005 |
| AP | 9.0 | 0.0081 | 3.0 | 0.0012 | 0.0 | 0.0002 | 0.0 | 0.0002 |

To further substantiate these observations, Table VIII presents the results of the Wilcoxon signed-rank test, which statistically validate the superiority of our model over competing approaches. Specifically, our model consistently achieves significantly better performance across all metrics (Accuracy, AUC, and AP) when compared to SlowFast, ViT, and both versions of GPT-4V+Prompt. The p-values, all below 0.05, underscore the robustness of these differences, particularly for metrics requiring fine-grained recognition such as AP. These results demonstrate that our model not only excels in general performance but also offers a statistically significant advantage in handling diverse interaction styles, further reinforcing its effectiveness as highlighted in Table VII.

Nevertheless, there remains room for improvement in recognizing certain categories, such as Complaint (CP), Parent Affection (Aff parent), Non-compliance (NC), and Praise (P). While our model achieves high accuracy for these categories, its sensitivity remains suboptimal. The primary reason for this limitation lies in the severe class imbalance within the dataset. Compared to majority classes like EA and S+, which account for 26.86% and 15.92% of the data, respectively, minority classes such as CP, MI, and NC comprise only 0.51%, 0.53%, and 0.55% of the dataset. The sample size of the majority classes exceeds that of the minority classes by more than 25 times, causing the model to adopt a more conservative decision-making approach and exhibit reluctance to predict positive outcomes for these underrepresented categories.

This limitation, however, is not unique to our model; it is a common challenge for all models. Notably, compared to mainstream video recognition models such as SlowFast and ViT, our model demonstrates superior performance on minority classes. For instance, our model achieves higher AUC and AP scores for categories representing less than 5% of the dataset, outperforming all other models. To address this issue further, we plan to collect additional data to improve the performance of model on minority classes. While data collection is a long-term process, we consider it a critical aspect of future research.

In summary, our model exhibits strong recognition capabilities and demonstrates greater robustness in handling imbalanced datasets compared to other mainstream models and the baseline. We aim to further enhance its performance by continuing to expand the dataset, ultimately striving for even better results in future studies.

### F. Ablation Study

1) Uni-modal recognition performance: To evaluate the effectiveness of the multimodal structure of the AV-FOS model, we decided to conduct ablation experiments by retraining and inferring the model with a single modality. This approach allowed us to observe whether the multimodal structure demonstrates improved performance across various metrics compared to single-modality structures. For the AV-FOS single-visual-modality perception model (V-FOS model), we removed the audio input and the audio processing modules such as Audio Tokenization and Audio Encoder 200, 212, 302, 310. However, we retained the Joint Encoder module, with the input token sequence for the Joint Encoder being only the visual tokens sequence processed by the Video Encoder, [Ev(tv)], instead of the concatenated multimodal token sequence [Ea(ta), Ev(tv)] used in the AV-FOS model. Similarly, for the AV-FOS single-audio-modality perception model (A-FOS model), we removed the visual input and perception modules, retaining the Joint Encoder module. The input token sequence for the Joint Encoder was the audio tokens sequence processed by the Audio Encoder, [Ea(ta)]. For both the A-FOS and V-FOS models, the subsequent Joint IS Decision Making Layers 342 (which is a neural network for deep learning) and other modules remained identical to those in the original AV-FOS model. To ensure a fair comparison, all pretrained modules (Audio Encoder, Visual Encoder, and Joint Encoder) in the A-FOS and V-FOS models underwent the same pretraining as those in the AV-FOS model. During the formal training on the FOS-II dataset, all training parameters (number of epochs, batch size, learning rate, etc.) and training and validation datasets were kept consistent with those used for the original AV-FOS model.

TABLE IX
THE ABLATION STUDY RESULT

| | | Fl | Strict | | | Time Cost |
|---|---|---|---|---|---|---|
| Strategy | Accuracy | Score | Accuracy | ACC | mAP | (Per Sample) |
| A-FOS (Audio) | 0.8523 | 0.5736 | 0.1912 | 08722 | 0.6542 | 0.0015 ± 0.0003 |
| V-FOS (Visual) | 0 8226 | 0.4917 | 0.1152 | 0.8296 | 0.5617 | 0.0009 ± 0.0003 |
| Without Pre-jam | 0 8322 | 0.5328 | 0.1382 | 08463 | 0.5630 | 0.0018 ± 0.0004 |
| Frame Aggregation | 08544 | 0.5853 | 0.1987 | 08881 | 0.6833 | 0.0055 ± 0.0015 |
| Crow-Frame Attention | 0.8407 | 0.5455 | 0.1521 | 0.8561 | 0.6879 | 0.0018 ± 0.0003 |
| Middle Frame Spatial Attention | 0 8517 | 0.5767 | 0.1918 | 08853 | 0.6749 | 0.0018 ± 0.0003 |
| Averaged Key Frame Attention | 0-8590 | 0.5936 | 0.2003 | 0.8868 | 0.6879 | 0.0018 ± 0.0003 |

The performance comparison between the two single-modality models, A-FOS and V-FOS, is presented in Table IX. It can be observed that the audio-based model, A-FOS, demonstrates stronger recognition capabilities than V-FOS, which aligns with the characteristics of our task. Most categories rely heavily on audio input, such as VI+ (Positive Vague Instruction), SI+ (Positive Specific Instruction), and Q+ (Positive Question). Additionally, even for instances that require visual modality for classification, such as EA (Engaged Activity of Play), audio cues (e.g., sounds from toy collisions) are often present. However, the incorporation of the visual modality further enhances the performance of multimodal perception models, resulting in the highest performance being achieved by our AV-FOS model.
2) Without CAV-MAE pretraining performance: To investigate the impact of the CAV-MAE pretraining strategy on our AV-FOS model, we conducted this ablation experiment. In this experiment, the model, training methods/hyperparameters, and dataset partitioning were identical to those of the original pretrained AV-FOS model. The only difference was that all parameters of the model were randomly initialized in this experiment. The results are presented in Table IX.

The results indicate that even without CAV-MAE pretraining, the AV-FOS model still performs very well, surpassing the baseline GPT4V with prompt engineering method. The accuracy exceeded 83%, demonstrating that the multimodal structure itself has strong performance even without relying on pretraining data. However, the overall performance was still inferior to the pretrained version of the AV-FOS model. While the accuracy showed a relatively small decrease (by 2%), the performance gap was more significant in metrics reflecting the model ability to handle data imbalance, such as F1 score and mAP. The AV-FOS model without pretraining showed a decrease of 6% in F1 score and 12% in mAP compared to the pretrained AV-FOS model. This suggests that pretraining can significantly enhance the model ability to handle data distribution imbalance and slightly improve its accuracy, which is highly beneficial for the model application in clinical settings.
3) Visual Temporal Information Perception Module: To enhance the model capability of perceiving visual temporal information, the system has two strategies: Cross-Frame Attention and Averaged Key Frame Attention. The experimental results demonstrate that the Averaged Key Frame Attention strategy outperforms both the Middle Frame Spatial Attention strategy (which lacks temporal information perception) and the Cross-Frame Attention strategy. This outcome aligns with our expectations. During the pretraining phase, we adopted the CAV-MAE framework, which involves training the model by randomly extracting a single frame from a video rather than using multiple frames. Consequently, the Cross-Frame Attention strategy is unable to fully leverage the prior knowledge learned during pretraining. This limitation results in its performance falling short of even the Middle Frame Spatial Attention strategy. In contrast, the Averaged Key Frame Attention strategy retains the original dimensions of each frame, averaging the pixel values across frames. This approach maximally exploits the pretraining knowledge while preserving some spatiotemporal information, leading to the best performance among the tested strategies. We also compared our approach to the Frame Aggregation strategy, which was originally in the CAV-MAE framework. This method involves performing inference three times using the first frame, middle frame, and last frame of a 10-second video clip, and then averaging the results. While Frame Aggregation achieves relatively high accuracy, it is extremely time-consuming. In contrast, the Averaged Key Frame Attention strategy not only outperforms Frame Aggregation in most metrics but also requires only a single inference step, reducing inference time to approximately one-third of that of Frame Aggregation. The tripling of inference time with Frame Aggregation poses a significant drawback, particularly in real-world deployment scenarios. Not all computers, especially those in clinical settings, can match the computational speed of our laboratory hardware. Slow inference times could hinder the algorithm clinical applicability. Therefore, considering both efficiency and effectiveness, the Averaged Key Frame Attention strategy emerges as the optimal choice for our model, striking a balance between computational feasibility and performance.

### G. Inference Visualization

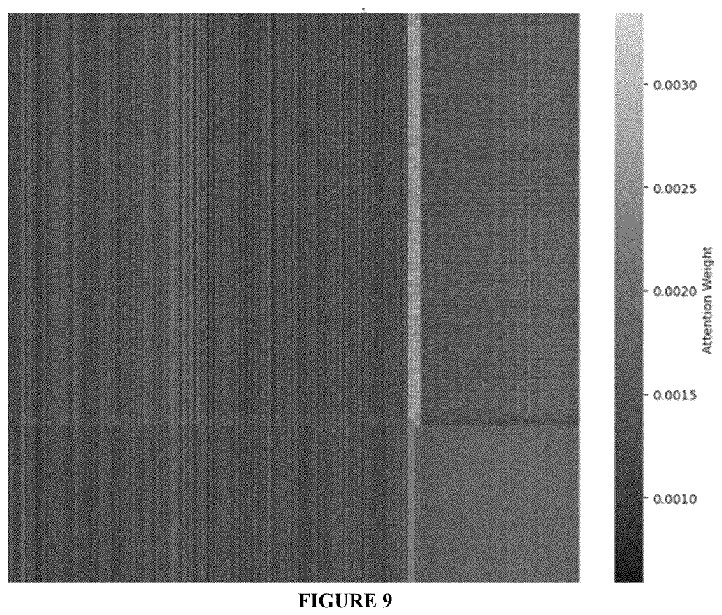

FIG. 9 illustrates the attention distribution within the fusion perception layer. The visualization reveals four distinct attention regions corresponding to:
1) Visual modality attending to the visual modality.
2) Visual modality attending to the audio modality.
3) Audio modality attending to the visual modality.
4) Audio modality attending to the audio modality. These patterns indicate that the model has effectively learned to differentiate the focus of attention based on the semantic relationships across different modalities. Specifically, the model demonstrates high sensitivity in cross-modal attention between visual and audito modalities, reflecting its capability to integrate information and model inter-modal relationships effectively. Meanwhile, the significant intra-modal attention weights highlight the robustness of the model in capturing modality-specific features. Collectively, these characteristics highlight the strong capacity of the model for multimodal perception and fusion tasks.

### V. CONCLUSION

To address the challenges in recognizing the complex behaviors and interactions of autistic children, thereby aiding in their diagnosis, symptom assessment/mitigation, and treatment, this study has: 1. a dataset based on the FOS behavior scale specifically for children with autism. This dataset was constructed from clinically collected data annotated by professionals with medical expertise. 2. Introduced a transformer-based deep learning model, AV-FOS, capable of automatically generating FOS-II scales from videos, which holds significant clinical value. This model can utilize self-supervised learning methods to pretrain on large-scale unlabeled video datasets unrelated to autism and make final FOS IS judgments based on both audio and video modalities, demonstrating high accuracy and robustness against imbalanced data. 3. Explored the application of large AI models and prompt engineering in the field of autism behavior recognition.

However, it should be noted that the current FOS-II dataset in this study has an insufficient amount of data for certain classes, i.e. unbalanced, which is not ideal for training deep learning models. Nonetheless, the data collection and annotation process have been structured in efficient manner, allowing for the collection and annotation of more data in the future, which will enable the training of more effective models. Additionally, the visual perception module of the AV-FOS model currently processes only a single frame from the original video, lacking the capability to recognize temporal information. This aspect can be optimized and improved in future work.

Furthermore, the manual annotation of datasets is time-consuming and labor-intensive, and privacy considerations for patients are paramount. Consequently, the academic community currently lacks high-quality public datasets for autistic children. Future research could greatly benefit from AI-generated video and audio data, automatically created based on the FOS-II scale or other behavioral scales. Such advancements would significantly contribute to the behavior analysis, diagnosis, and treatment for the autistic children.

The documents mentioned herewith form a part of the application and are incorporated herein by reference. In addition to the embodiments shown and described, the system and method of the disclosure can be implemented by a computer or computing device having a processor, processing device or controller to perform various functions and operations in accordance with the disclosure, including but not limited to the pretraining system (FIG. 5) and the AV-FOS system (FIG. 6). The computer can be, for instance, a personal computer (PC), server or mainframe computer. The processor may also be provided with one or more of a wide variety of components or subsystems including, for example, a co-processor, graphic processing unit (GPU), tensor-processing unit (TPU), register, data processing devices and subsystems, wired or wireless communication links, input devices, monitors, memory or storage devices such as a database. All or parts of the system and processes can be stored on or read from computer-readable media. The system can include computer-readable medium, such as a hard disk, having stored thereon machine executable instructions for performing the processes described. All or parts of the system, processes, and/or data utilized in the disclosure can be stored on or read from the storage device(s). The storage device(s) can have stored thereon machine executable instructions for performing the processes of the disclosure. The processing device can execute software that can be stored on the storage device.

The system and method of the disclosure can also be implemented by or on a non-transitory computer readable medium, such as any tangible medium that can store, encode or carry non-transitory instructions for execution by the computer and cause the computer to perform any one or more of the operations of the disclosure described herein, or that is capable of storing, encoding, or carrying data structures utilized by or associated with instructions.

As used herein, when an element or feature is described as being “configured,” that element or feature is structurally arranged or formed to accomplish the stated purpose. As used with respect to a processing device (e.g., computer), the term “configured” means that the processing device is structurally arranged or ordered (e.g., by supplying, arranging or connecting a specific set of internal or external components or modules, for example that perform certain operations) to accomplish the stated purpose or task.

The description and drawings of the present disclosure provided in the paper should be considered as illustrative only of the principles of the disclosure. The disclosure may be configured in a variety of ways and is not intended to be limited by the disclosed embodiment. Numerous applications of the disclosure will readily occur to those skilled in the art. Therefore, it is not desired to limit the disclosure to the specific examples disclosed or the exact construction and operation shown and described. Rather, all suitable modifications and equivalents may be resorted to, falling within the scope of the disclosure.

The following citations are hereby incorporated by reference in their entireties.
[1] M. Lee and K. Chung, “Development of Parent Child Interaction-Direct Observation Checklist (PCI-D) for Children with Developmental Disabilities,” *Journal of Rehabilitation Psychology*, vol. 23, no. 2, pp. 367+.
[2] Y. Gong, A. Rouditchenko, A. H. Liu, D. Harwath, L. Karlinsky, H. Kuehne, and J. Glass, “Contrastive audio-visual masked autoencoder,” arXiv preprint arXiv:2210.07839, 2022.
[3] G. Bertasius, H. Wang, and L. Torresani, “Is space-time attention all you need for video understanding?,” in *Proc. ICML*, vol. 2, no. 3, p. 4, 2021.
[4] R. Karim and R. P. Wildes, “Understanding Video Transformers for Segmentation: A Survey of Application and Interpretability,” arXiv preprint arXiv:2310.12296, 2023.
[5] OpenAI, “GPT-4V System Card,” 2023. [Online]. Available: https://openai.com/index/gpt-4v-system-card/. [Accessed: May 22, 2024].
[6] C. Feichtenhofer, H. Fan, J. Malik, and K. He, “SlowFast Networks for Video Recognition,” in *Proceedings of the IEEE/CVF International Conference on Computer Vision (ICCV)*, October 2019.
[7] A. Dosovitskiy, L. Beyer, A. Kolesnikov, D. Weissenborn, X. Zhai, T. Unterthiner, M. Dehghani, M. Minderer, G. Heigold, S. Gelly, et al., “An image is worth 16×16 words: Transformers for image recognition at scale,” arXiv preprint arXiv:2010.11929, 2020.
[8] M. R. Sanders, C. Markie-Dadds, L. A. Tully, and W. Bor, “The triple P-positive parenting program: a comparison of enhanced, standard, and self-directed behavioral family intervention for parents of children with early onset conduct problems,” *Journal of Consulting and Clinical Psychology*, vol. 68, no. 4, p. 624, 2000.
[9] D. S. Pasalich, M. R. Dadds, and D. J. Hawes, “Cognitive and affective empathy in children with conduct problems: additive and interactive effects of callous-unemotional traits and autism spectrum disorders symptoms,” *Psychiatry Research*, vol. 219, no. 3, pp. 625-630, November 2014.
[10] M.-I. Georgescu, E. Fonseca, R. T. Ionescu, M. Lucic, C. Schmid, and A. Arnab, “Audiovisual masked autoencoders,” in *Proceedings of the IEEE/CVF International Conference on Computer Vision*, pp. 16144-16154, 2023.
[11] A. Ali, F. F. Negin, F. F. Bremond, and S. Thümmler, “Video-based Behavior Understanding of Children for Objective Diagnosis of Autism,” in *VISAPP 2022-17th International Conference on Computer Vision Theory and Applications*, Online, France, February 2022.
[12] S. Weigelt, K. Koldewyn, and N. Kanwisher, “Face identity recognition in autism spectrum disorders: A review of behavioral studies,” *Neuroscience & Biobehavioral Reviews*, vol. 36, no. 3, pp. 1060-1084, 2012.
[13] M. Cheng, Y. Zhang, Y. Xie, Y. Pan, X. Li, W. Liu, C. Yu, D. Zhang, Y. Xing, X. Huang, et al., “Computer-aided autism spectrum disorder diagnosis with behavior signal processing,” *IEEE Transactions on Affective Computing*, 2023.
[14] J. Han, G. Jiang, G. Ouyang, and X. Li, “A Multimodal Approach for Identifying Autism Spectrum Disorders in Children,” *IEEE Transactions on Neural Systems and Rehabilitation Engineering*, vol. 30, pp. 2003-2011, 2022.
[15] H. Nori, N. King, S. M. Mckinney, D. Carignan, and E. Horvitz, “Capabilities of GPT-4 on medical challenge problems,” arXiv preprint arXiv:2303.13375, 2023.
[16] J. Achiam, S. Adler, S. Agarwal, L. Ahmad, I. Akkaya, F. L. Aleman, D. Almeida, J. Altenschmidt, S. Altman, S. Anadkat, et al., “GPT-4 technical report,” arXiv preprint arXiv:2303.08774, 2023.
[17] Z. Lian, L. Sun, H. Sun, K. Chen, Z. Wen, H. Gu, B. Liu, and J. Tao, “GPT-4V with emotion: A zero-shot benchmark for Generalized Emotion Recognition,” *Information Fusion*, vol. 108, p. 102367, 2024.
[18] S. Dhingra, M. Singh, V. S. B., N. Malviya, and S. S. Gill, “Mind meets machine: Unravelling GPT-4's cognitive psychology,” *BenchCouncil Transactions on Benchmarks, Standards and Evaluations*, vol. 3, no. 3, p. 100139, 2023.
[19] A. Vaswani, N. Shazeer, N. Parmar, J. Uszkoreit, L. Jones, A. N. Gomez, Ł. Kaiser, and I. Polosukhin, “Attention is all you need,” in *Advances in Neural Information Processing Systems*, vol. 30 Curran Associates, Inc., 2017. [Online]. Available: https://proceedings.neurips.cc/paper_files/paper/2017/file/3f5ee243547dee91fbd053c1c4a845aa-Paper.pdf
[20] K. He, X. Chen, S. Xie, Y. Li, P. Dollar, and R. Girshick, “Masked Autoencoders Are Scalable Vision Learners,” in *Proceedings of the IEEE/CVF Conference on Computer Vision and Pattern Recognition (CVPR)*, pp. 16000-16009, June 2022.
[21] J. F. Gemmeke, D. P. W. Ellis, D. Freedman, A. Jansen, W. Lawrence, R. C. Moore, M. Plakal, and M. Ritter, “Audio Set: An ontology and human-labeled dataset for audio events,” in *2017 IEEE International Conference on Acoustics, Speech and Signal Processing (ICASSP)*, pp. 776-780, 2017, doi: 10.1109/ICASSP.2017.7952261.
[22] OpenAI, “Enterprise Privacy,” 2024. [Online]. Available: https://openai.com/enterprise-privacy/. [Accessed: Jun. 27, 2024].
[23] W. Kay, J. Carreira, K. Simonyan, B. Zhang, C. Hillier, S. Vijayanarasimhan, F. Viola, T. Green, T. Back, P. Natsev, et al., “The kinetics human action video dataset,” arXiv preprint arXiv:1705.06950, 2017.
[24] T. Ridnik, E. Ben-Baruch, A. Noy, and L. Zelnik-Manor, “Imagenet-21k pretraining for the masses,” arXiv preprint arXiv:2104.10972, 2021.
[25] O. Russakovsky, J. Deng, H. Su, J. Krause, S. Satheesh, S. Ma, Z. Huang, A. Karpathy, A. Khosla, M. Bernstein, A. C. Berg, and L. Fei-Fei, “ImageNet Large Scale Visual Recognition Challenge,” *International Journal of Computer Vision (UCV)*, vol. 115, no. 3, pp. 211-252, 2015, doi: 10.1007/s11263-015-0816-y.

1. A behavior recognition system for analyzing an audio-video signal to detect challenging behaviors in autism via behavioral features, said system comprising:
a processor configured to:
segment the audio-video signal into clips of audio data and video data, each of said clips having a predefined duration and annotated with interaction styles;
sample and preprocess the audio data and video data of said clips to provide square video patches and square audio patches;
tokenize the square video patches to embed video positional information and video modality information, and tokenize the square audio patches to embed audio positional information and audio modality information; and
predict behaviors based on the tokenized square video patches and the tokenized square audio patches.

2. The system of claim 1, wherein the sample and preprocess of the video data comprises selecting a key frame, resizing and center cropping the key frame, and segment the key frame into the square video patches.

3. The system of claim 1, wherein the sample and preprocess of the audio data comprises converting the audio data into spectrograms, sampling the spectrograms to provide a sequence of features, and segmenting the spectrograms into the square audio patches.

4. The system of claim 1, wherein said key fame uses both reconstruction loss and contrastive loss.

5. The system of claim 1, wherein said system predicts behavioral features with an 80-90% accuracy.
